(19) United States
(12) Patent Application Publication
(10) Pub. No.: US 2005/0020494 A1
(43) Pub. Date: Jan. 27, 2005

(54) Modified human growth hormone

(76) Inventors: Francis J Carr, Balmedie (GB); Graham Carter, By Newmachar (GB)

Correspondence Address: Talivaldis Cepuritis;Olson & Hierl Ltd, 36th Floor, 20 North Wacker Drive, Chicago, IL, 60606, US

(21) Appl. No.: 10/488,662

(22) PCT Filed: Aug. 30, 2002
(86) PCT No.: PCT/EP02/09716

(30) Foreign Application Priority Data
Sep. 4, 2001 (EP) 01121153.9

(51) Int. Cl. A61K 38/27

(52) U.S. Cl. 514/12; 530/399

(57) ABSTRACT

The invention relates to the modification of human growth hormone (high) to result in human growth hormone proteins that are substantially non-immunogenic or less immunogenic than any non-modified counterpart when used in-vivo. The invention relates, furthermore, to T-cell epitome sequences deriving from high, which are immunogenic.

### FIELD OF THE INVENTION

The present invention relates to polypeptides to be administered especially to humans and in particular for therapeutic use. The polypeptides are modified polypeptides whereby the modification results in a reduced propensity for the polypeptide to elicit an immune response upon administration to the human subject. The invention in particular relates to the modification of human growth hormone to result in human growth hormone proteins that are substantially non-immunogenic or less immunogenic than any non-modified counterpart when used in vivo.

### BACKGROUND OF THE INVENTION

There are many instances whereby the efficacy of a therapeutic protein is limited by an unwanted immune reaction to the therapeutic protein. Several mouse monoclonal antibodies have shown promise as therapies in a number of human disease settings but in certain cases have failed due to the induction of significant degrees of a human anti-murine antibody (HAMA) response [Schroff, R. W. et al (1985) Cancer Res. 45: 879-885; Shawler, D.L. et al (1985) J. Immunol. 135: 1530-1535]. For monoclonal antibodies, a number of techniques have been developed in attempt to reduce the HAMA response [WO 89/09622; EP 0239400; EP 0438310; WO 91/06667]. These recombinant DNA approaches have generally reduced the mouse genetic information in the final antibody construct whilst increasing the human genetic information in the final construct. Notwithstanding, the resultant “humanized” antibodies have, in several cases, still elicited an immune response in patients [Issacs J. D. (1990) Sem. Immunol. 2: 449, 456; Rebello, P. R. et al (1999) Transplantation 68: 1417-1420].

Antibodies are not the only class of polypeptide molecule administered as a therapeutic agent against which an immune response may be mounted. Even proteins of human origin and with the same amino acid sequences as occur within humans can still induce an immune response in humans. Notable examples amongst others include the therapeutic use of granulocyte-macrophage colony stimulating factor [Wadhwa, M. et al (1999) Clin. Cancer Res. 5: 1353-1361] and interferon alpha 2 [Russo, D. et al (1996) Bri. J. Haem. 94: 300-305; Stein, R. et al (1988) New Engl. J. Med. 318: 1409-1413]. In such situations where these human proteins are immunogenic, there is a presumed breakage of immunological tolerance that would otherwise have been operating in these subjects to these proteins.

This situation is different where the human protein is being administered as a replacement therapy for example in a genetic disease where there is a constitutional lack of the protein such as can be the case for diseases such as hemophilia A, hemophilia B, Gauchers disease and numerous other examples. In such cases, the therapeutic replacement protein may function immunologically as a foreign molecule from the outset, and where the individuals are able to mount an immune response to the therapeutic, the efficacy of the therapy is likely to be significantly compromised.

Irrespective of whether the protein therapeutic is seen by the host immune system as a foreign molecule, or if an existing tolerance to the molecule is overcome, the mechanism of immune reactivity to the protein is the same. Key to the induction of an immune response is the presence within the protein of peptides that can stimulate the activity of T-cells via presentation on MHC class II molecules, so-called “T-cell epitopes”. Such T-cells epitopes are commonly defined as any amino acid residue sequence with the ability to bind to MHC Class II molecules. Implicitly, a “T-cell epitope” means an epitope which when bound to MHC molecules can be recognized by a T-cell receptor (TCR), and which can, at least in principle, cause the activation of these T-cells by engaging a TCR to promote a T-cell response.

MHC Class II molecules are a group of highly polymorphic proteins which play a central role in helper T-cell selection and activation. The human leukocyte antigen group DR (HLA-DR) are the predominant isotype of this group of proteins however, isotypes HLA-DQ and HLA-DP perform similar functions. In the human population, individuals bear two to four DR alleles, two DQ and two DP alleles. The structure of a number of DR molecules has been solved and these appear as an open-ended peptide binding groove with a number of hydrophobic pockets which engage hydrophobic residues (pocket residues) of the peptide [Brown et al Nature (1993) 364: 33; Stern et al (1994) Nature 368: 215]. Polymorphism identifying the different allotypes of class II molecule contributes to a wide diversity of different binding surfaces for peptides within the peptide binding grove and at the population level ensures maximal flexibility with regard to the ability to recognise foreign proteins and mount an immune response to pathogenic organisms.

An immune response to a therapeutic protein proceeds via the MHC class II peptide presentation pathway. Here exogenous proteins are engulfed and processed for presentation in association with MHC class II molecules of the DR, DQ or DP type. MHC Class II molecules are expressed by professional antigen presenting cells (APCs), such as macrophages and dendritic cells amongst others. Engagement of a MHC class II peptide complex by a cognate T-cell receptor on the surface of the T-cell, together with the cross-binding of certain other co-receptors such as the CD4 molecule, can induce an activated state within the T-cell. Activation leads to the release of cytokines further activating other lymphocytes such as B cells to produce antibodies or activating T killer cells as a full cellular immune response.

T-cell epitope identification is the first step to epitope elimination, however there are few clear cases in the art where epitope identification and epitope removal are integrated into a single scheme. Thus WO98/52976 and WO00/34317 teach computational threading approaches to identifying polypeptide sequences with the potential to bind a sub-set of human MHC class II DR allotypes. In these teachings, predicted T-cell epitopes are removed by the use of judicious amino acid substitution within the protein of interest. However with this scheme and other computationally based procedures for epitope identification [Godkin, A. J. et al (1998) J. Immunol. 161: 850-858; Sturniolo, T. et al (1999) Nat. Biotechnol. 17: 555-561], peptides predicted to be able to bind MHC class II molecules may not function as T-cell epitopes in all situations, particularly, in vivo due to the processing pathways or other phenomena.

Equally, in vitro methods for measuring the ability of synthetic peptides to bind MHC class II molecules, for example using B-cell lines of defined MHC allotype as a source of MHC class II binding surface and may be applied to MHC class II ligand identification [Marshall K. W. et al. (1994) J. Immunol 152:4946-4956; O'Sullivan et al (1990) J. Immunol. 145: 1799-1808; Robadey C. et al (1997) J. Immunol 159: 3238-3246]. However, such techniques are not adapted for the screening multiple potential epitopes to a wide diversity of MHC allotypes, nor can they confirm the ability of a binding peptide to function as a T-cell epitope.

Recently techniques exploiting soluble complexes of recombinant MHC molecules in combination with synthetic peptides have come into use [Kern, F. et al (1998) Nature Medicine 4:975-978; Kwok, W. W. et al (2001) TRENDS in Immunol. 22:583-588]. These reagents and procedures are used to identify the presence of T-cell clones from peripheral blood samples from human or experimental animal subjects that are able to bind particular MHC-peptide complexes and are not adapted for the screening multiple potential epitopes to a wide diversity of MHC allotypes.

Biological assays of T-cell activation provide a practical option to providing a reading of the ability of a test peptide/protein sequence to evoke an immune response. Examples of this kind of approach include the work of Petra et al using T-cell proliferation assays to the bacterial protein staphylokinase, followed by epitope mapping using synthetic peptides to stimulate T-cell lines [Petra, A. M. et al (2002) J. Immunol. 168: 155-161]. Similarly, T-cell proliferation assays using synthetic peptides of the tetanus toxin protein have resulted in definition of immunodominant epitope regions of the toxin [Reece J. C. et al (1993) J. Immunol. 151: 6175-6184]. WO99/53038 discloses an approach whereby T-cell epitopes in a test protein may be determined using isolated sub-sets of human immune cells, promoting their differentiation in vitro and culture of the cells in the presence of synthetic peptides of interest and measurement of any induced proliferation in the cultured T-cells. The same technique is also described by Stickler et al [Stickler, M. M. et al (2000) J. Immunotherapy 23:654-660], where in both instances the method is applied to the detection of T-cell epitopes within bacterial subtilisin. Such a technique requires careful application of cell isolation techniques and cell culture with multiple cytoline supplements to obtain the desired immune cell sub-sets (dendritic cells, CD4+and or CD8+ T-cells) and is not conducive to rapid through-put screening using multiple donor samples.

As depicted above and as consequence thereof, it would be desirable to identify and to remove or at least to reduce T-cell epitopes from a given in principal therapeutically valuable but originally immunogenic peptide, polypeptide or protein. One of these potential therapeutically valuable molecules is human growth hormone (herein abbreviated to hGH).

Natural hGH is a pituitary hormone of 22 kDa molecular weight and 191 amino acid residues. An alternative 20 kDa product derived by alternative splicing is also recognised and has some altered properties compared to the 22 kDa form [Wada, M. et al (1997) Mol. Cell Endocrinol. 133: 99-107]. The 22 kDa protein been produced using recombinant techniques in a variety of host organisms including E.coli [Goeddel, D. et al (1979) Nature 281: 544-548] Bacillus subtilis [Honjo, J. et al (1987) J. Biotech 6: 191-204], yeast [Hiramatsu, R. et al (1991) Appl. Environ. Microbiol. 57: 2052-2056] and animal cells [Lupker, J. et al (1983) Gene 24: 281-287]. Pharmaceutical preparations of hGH are used for the treatment of pituitary dwarfism, paediatric chronic renal failure and similar indications. In addition to its ability to promote growth, the protein has a variety of biological activities including activation of macrophages and insulin like effects [Chawler, R. (1993) Ann. Rev. Med. 34: 519; Edwards, C. et al (1988) Science 239: 769].

The present invention is concerned with human growth hormone (hGH) and the amino acid sequence of the secreted form of the hGH protein depicted in single-letter code is as follows:
FPTIPLSRLFQNAMLRAHRLHQLAFDTYEEFEEAYIPKEQKYSFLQAPQASLCFSESIPTPSNRE QAQQKSNLQLLRISLLLIQSWLEPVGFLRSVFANSLVYGASDSDVYDLLKDLEEGIQTLMGRLED GSPRTGQAFKQTYAKFDANSHNDDALLKNYGLLYCFRKDMDKVETFLRIVQCRSVEGSCGF

It is a particular objective of the present invention to provide modified hGH proteins in which the immune characteristic is modified by means of reduced numbers of potential T-cell epitopes.

Others have provided hGH molecules including modified hGH and schemes for its recombinant production, purification and therapeutic use [EP 0107890, U.S. Pat. No. 4,517,181, EP 0105759; U.S. Pat. No. 4,703,035; U.S. Pat. No. 4,658,021; EP0022242; EP0001929; EP0001939; U.S. Pat. No. 4,342,832; U.S. Pat. No. 4,601,980; U.S. Pat. No. 4,604,359; U.S. Pat. No. 4,634,677; U.S. Pat. No. 4,898,830; U.S. Pat. No. 5,424,119; U.S. Pat. No. 4,366,246; U.S. Pat. No. 4,425,437; U.S. Pat. No. 4,431,739; U.S. Pat No. 4,563,424; U.S. Pat. No. 4,571,421; EP 0131843; EP 0319049; U.S. Pat. No. 4,831,120; U.S. Pat. No. 4,871,835; U.S. Pat. No. 4,997,916; U.S. Pat. No. 5,612,315; U.S. Pat. No.5,633,352; U.S. Pat. No. 5,618,697; U.S. Pat. No. 5,635,604; EP 0127658; EP 0217814; U.S. Pat. No. 5,898,030; EP0804223] but these teachings do not address the importance of T cell epitopes to the immunogenic properties of the protein nor have been conceived to directly influence said properties in a specific and controlled way according to the scheme of the present invention. An example in this regard is provided by Lowman and Wells [Lowman H. B. & Wells J. A. (1993) J. Mol. Biol. 243: 564-578] who have used phage display in the creation of a hGH variant which exhibits an approximately 400-fold increased binding affinity for the hGH receptor. This high affinity variant contains fifteen amino acid substitutions but no consideration of immunological properties of the new variant molecule has been made.

However, as disclosed for the first time herein below, the present inventors have discovered that of these fifteen substitutions in the high affinity variant, seven (at positions 10, 14, 42, 45, 54, 176 and 179) can be expected to provide immunological benefit according to the scheme of the present invention.

It is highly desired to provide hGH with reduced or absent potential to induce an immune response in the human subject.

### SUMMARY AND DESCRIPTION OF THE INVENTION

The present invention provides for modified forms of hGH, in which the immune characteristic is modified by means of reduced or removed numbers of potential T-cell epitopes.

The invention discloses sequences identified within the hGH primary sequence that are potential T-cell epitopes by virtue of MHC class II binding potential. This disclosure specifically pertains the human hGH protein sequence given above herein and comprising 191 amino acid residues.

The present invention discloses the major regions of the hGH primary sequence that are immunogenic in man and thereby provides the critical information required to conduct modification to the sequences to eliminate or reduce the immunogenic effectiveness of these sites.

In one embodiment, synthetic peptides comprising the immunogenic regions can be provided in pharmaceutical composition for the purpose of promoting a tolerogenic response to the whole molecule.

In a further embodiment hGH molecules modified within the epitope regions herein disclosed can be used in pharmaceutical compositions.

In summary the invention relates to the following issues:
a modified molecule having the biological activity of hGH and being substantially non-immunogenic or less immunogenic than any non-modified molecule having the same biological activity when used in vivo;
an accordingly specified molecule, wherein said loss of immunogenicity is achieved by removing one or more T-cell epitopes derived from the originally non-modified molecule;
an accordingly specified molecule, wherein said loss of immunogenicity is achieved by reduction in numbers of MHC allotypes able to bind peptides derived from said molecule;
an accordingly specified molecule, wherein one T-cell epitope is removed;
an accordingly specified molecule, wherein said originally present T-cell epitopes are MHC class II ligands or peptide sequences which show the ability to stimulate or bind T-cells via presentation on class II;
an accordingly specified molecule, wherein said peptide sequences are selected from the group as depicted in Table 1;
an accordingly specified molecule, wherein 1-9 amino acid residues preferably one amino acid residue in any of the originally present T-cell epitopes are altered;
an accordingly specified molecule, wherein the alteration of the amino acid residues is substitution, addition or deletion of originally present amino acid(s) residue(s) by other amino acid residue(s) at specific position(s);
an accordingly specified molecule, wherein one or more of the amino acid residue substitutions are carried out as indicated in Table 2;
an accordingly specified molecule, wherein (additionally) one or more of the amino acid residue substitutions are carried out as indicated in Table 3 for the reduction in the number of MHC allotypes able to bind peptides derived from said molecule;
an accordingly specified molecule, wherein, if necessary, additionally further alteration usually by substitution, addition or deletion of specific amino acid(s) is conducted to restore biological activity of said molecule;
an accordingly specified hGH molecule, wherein one or more of the amino acid substitutions is conducted at a position corresponding to any of the amino acids specified within Tables 2 or 3;
an accordingly specified hGH molecule, wherein one or more of the amino acid substitutions is conducted at a position corresponding to any of the amino acids specified within Tables 2 or 3 but excluding any of those substitutions known from the record of hGH genetic mutations to be incompatible with functional protein;
a pharmaceutical composition comprising any of the peptides or modified peptides of above having the activity of binding to MHC class II;
a DNA sequence or molecule which codes for any of said specified modified molecules as defined above and below;
a pharmaceutical composition comprising a modified molecule having the biological activity of hGH as defined above and/or in the claims, optionally together with a pharmaceutically acceptable carrier, diluent or excipient;
a method for manufacturing a modified molecule having the biological activity of hGH as defined in any of the claims of the above-cited claims comprising the following steps: (i) determining the amino acid sequence of the polypeptide or part thereof; (ii) identifying one or more potential T-cell epitopes within the amino acid sequence of the protein by any method including determination of the binding of the peptides to MHC molecules using in vitro or in silico techniques or biological assays; (iii) designing new sequence variants with one or more amino acids within the identified potential T-cell epitopes modified in such a way to substantially reduce or eliminate the activity of the T-cell epitope as determined by the binding of the peptides to MHC molecules using in vitro or in silico techniques or biological assays; (iv) constructing such sequence variants by recombinant DNA techniques and testing said variants in order to identity one or more variants with desirable properties; and (v) optionally repeating steps (ii)-(iv);
an accordingly specified method, wherein step (iii) is carried out by substitution, addition or deletion of 1-9 amino acid residues in any of the originally present T-cell epitopes;
an accordingly specified method, wherein the alteration is made with reference to an homologous protein sequence and/or in silico modeling techniques;
an accordingly specified method, wherein step (ii) of above is carried out by the following steps: (a) selecting a region of the peptide having a known amino acid residue sequence; (b) sequentially sampling overlapping amino acid residue segments of predetermined uniform size and constituted by at least three amino acid residues from the selected region; (c) calculating MHC Class II molecule binding score for each said sampled segment by summing assigned values for each hydrophobic amino acid residue side chain present in said sampled amino acid residue segment; and (d) identifying at least one of said segments suitable for modification, based on the calculated MHC Class II molecule binding score for that segment, to change overall MHC Class II binding score for the peptide without substantially reducing therapeutic utility of the peptide; step (c) is preferably carried out by using a Böhm scoring function modified to include 12-6 van der Waal's ligand-protein energy repulsive term and ligand conformational energy term by (1) providing a first data base of MHC Class II molecule models; (2) providing a second data base of allowed peptide backbones for said MHC Class II molecule models; (3) selecting a model from said first data base; (4) selecting an allowed peptide backbone from said second data base; (5) identifying amino acid residue side chains present in each sampled segment; (6) determining the binding affinity value for all side chains present in each sampled segment; and repeating steps (1) through (5) for each said model and each said backbone;
a 13mer T-cell epitope peptide having a potential MHC class II binding activity and created from non-modified hGH, selected from the group as depicted in Table 1 and its use for the manufacture of hGH having substantially no or less immunogenicity than any non-modified molecule with the same biological activity when used in vivo;
a peptide sequence consisting of at least 9 consecutive amino acid residues of a 13mer T-cell epitope peptide as specified above and its use for the manufacture of hGH having substantially no or less immunogenicity than any non-modified molecule with the same biological activity when used in vivo;
using a panel of synthetic peptides in a biological T-cell assay to map the immunogenic region(s) of human hGH;
using a panel of hGH protein variants in a biological T-cell assay to select variants displaying minimal immunogenicity in vitro;
using a panel of synthetic peptide variants in a biological T-cell assay to select peptide sequences displaying minimal immunogenicity in vitro;
using biological assays of T-cell stimulation to select a protein variant which exhibits a stimulation index of less than 2.0 and preferably less than 1.8 in a naïve T-cell assay;
construction of a T-cell epitope map of hGH protein using PBMC isolated from healthy donors and a screening method involving the steps comprising: i) antigen priming in vitro using synthetic peptide or whole protein immunogen for a culture period of up to 7 days; ii) addition of IL-2 and culture for up to 3 days; iii) addition of primed T cells to autologous irradiated PBMC and re-challenge with antigen for a further culture period of 4 days and iv) measurement of proliferation index by any suitable method;
hGH derived peptide sequences able to evoke a stimulation index of greater than 1.8 and preferably greater than 2.0 in a naïve T-cell assay;
hGH derived peptide sequences having a stimulation index of greater than 1.8 and preferably greater than 2.0 in a naïve T-cell assay wherein the peptide is modified to a minimum extent and tested in the naïve T-cell assay and found to have a stimulation index of less than 2.0;
hGH derived peptide sequences sharing 100% amino acid identity with the wild-type protein sequence and able to evoke a stimulation index of 1.8 or greater and preferably greater than 2.0 in a T-cell assay;
an accordingly specified hGH peptide sequence modified to contain less than 100% amino acid identity with the wild-type protein sequence and evoking a stimulation index of less than 2.0 when tested in a T-cell assay;
a hGH molecule containing a modified peptide sequence which when individually tested evokes a stimulation index of less than 2.0 in a T-cell assay,
a hGH molecule containing modifications such that when tested in a T-cell assay evokes a reduced stimulation index in comparison to a non modified protein molecule;
a hGH molecule in which the immunogenic regions have been mapped using a T-cell assay and then modified such that upon re-testing in a T-cell assay the modified protein evokes a stimulation index smaller than the parental (non-modified) molecule and most preferably less than 2.0.

The term “T-cell epitope” means according to the understanding of this invention an amino acid sequence which is able to bind MHC class II, able to stimulate T-cells and/or also to bind (without necessarily measurably activating) T-cells in complex with MHC class II.

The term “peptide” as used herein and in the appended claims, is a compound that includes two or more amino acids. The amino acids are linked together by a peptide bond (defined herein below). There are 20 different naturally occurring amino acids involved in the biological production of peptides, and any number of them may be linked in any order to form a peptide chain or ring. The naturally occurring amino acids employed in the biological production of peptides all have the L-configuration. Synthetic peptides can be prepared employing conventional synthetic methods, utilizing L-amino acids, D-amino acids, or various combinations of amino acids of the two different configurations. Some peptides contain only a few amino acid units. Short peptides, e.g., having less than ten amino acid units, are sometimes referred to as “oligopeptides”. Other peptides contain a large number of amino acid residues, e.g. up to 100 or more, and are referred to as “polypeptides”. By convention, a “polypeptide” may be considered as any peptide chain containing three or more amino acids, whereas a “oligopeptide” is usually considered as a particular type of “short” polypeptide. Thus, as used herein, it is understood that any reference to a “polypeptide” also includes an oligopeptide. Further, any reference to a “peptide” includes polypeptides, oligopeptides, and proteins. Each different arrangement of amino acids forms different polypeptides or proteins. The number of polypeptides—and hence the number of different proteins—that can be formed is practically unlited. “Alpha carbon (Cα)” is the carbon atom of the carbon-hydrogen (CH) component that is in the peptide chain. A “side chain” is a pendant group to Cα that can comprise a simple or complex group or moiety, having physical dimensions that can vary significantly compared to the dimensions of the peptide.

The invention may be applied to any hGH species of molecule with substantially the same primary amino acid sequences as that disclosed herein and would include therefore hGH molecules derived by genetic engineering means or other processes and may contain more or less than 191 amino acid residues. Many of the peptide sequences of the present disclosure are in common with peptide sequences derived from hGH proteins of non-human origin or are at least substantially the same as those from non-human hGH proteins. Such protein sequences equally therefore fall under the scope of the present invention.

The invention is conceived to overcome the practical reality that soluble proteins introduced with therapeutic intent in man trigger an immune response resulting in development of host antibodies that bind to the soluble protein. The present invention seeks to address this by providing hGH proteins with altered propensity to elicit an immune response on administration to the human host. According to the methods described herein, the inventors have discovered the regions of the hGH molecule comprising the critical T-cell epitopes driving the immune responses to this protein.

The general method of the present invention leading to the modified hGH comprises the following steps:
(a) determining the amino acid sequence of the polypeptide or part thereof;
(b) identifying one or more potential T-cell epitopes within the amino acid sequence of the protein by any method including determination of the binding of the peptides to MHC molecules using in vitro or in silico techniques or biological assays;
(c) designing new sequence variants with one or more amino acids within the identified potential T-cell epitopes modified in such a way to substantially reduce or eliminate the activity of the T-cell epitope as determined by the binding of the peptides to MHC molecules using in vitro or in silico techniques or biological assays. Such sequence variants are created in such a way to avoid creation of new potential T-cell epitopes by the sequence variations unless such new potential T-cell epitopes are, in turn, modified in such a way to substantially reduce or eliminate the activity of the T-cell epitope; and
(d) constructing such sequence variants by recombinant DNA techniques and testing said variants in order to identify one or more variants with desirable properties according to well known recombinant techniques.

The identification of potential T-cell epitopes according to step (b) can be carried out according to methods describes previously in the art. Suitable methods are disclosed in WO 98/59244; WO 98/52976; WO 00/34317 and may preferably be used to identify binding propensity of hGH-derived peptides to an MHC class II molecule.

Another very efficacious method for identifying T-cell epitopes by calculation is described in the Example 1 which is a preferred embodiment according to this invention.

The results of an analysis according to step (b) of the above scheme and pertaining to the human hGH protein sequence is presented in Table 1.

TABLE 1

Peptide sequences in human hGH with potential
human MHC class II binding activity.

PTIPLSRLFQNAM, IPLSRLFQNAMLR, SRLFQNAMLRAHR,
RLFQNAMLRAHRL, NAMLRAHRLHQLA, AMLRAHRLRQLAF,
HRLHQLAFDTYEE, HQLAFDTYEEFEE, LAFDTYEEFEEAY,
DTYEEFEEAYIPK, EEFEEAYIPKEQK, EAYIPKEQKYSFL,
AYIPKEQKYSFLQ, QKYSFLQAPQASL, YSFLQAPQASLCF,
SFLQAPQASLCFS, ASLCFSESIPTPS, LCFSESIPTPSNR,
ESIPTPSNREQAQ, SNLQLLRISLLLI, LQLLRISLLLIQS,
QLLRISLLLIQSW, LRISLLLIQSWLE, ISLLLIQSWLEPV,
SLLLIQSWLEPVG, LLLIQSWLEPVGF, LLIQSWLEPVGFL,
QSWLEPVGFLRSV, SWLEPVGFLRSVF, EPVGFLRSVFANS,
VGFLRSVFANSLV, GFLRSVFANSLVY, RSVFANSLVYGAS,
SVFANSLVYGASD, NSLVYGASDSDVY, SLVYGASDSDVYD,
LVYGASDSDVYDL, SDVYDLLKDLEEG, DVYDLLKDLEEGI,
YDLLKDLEEGIQT, DLLKDLEEGIQTL, KDLEEGIQTLMGR,
EGIQTLMGRLEDG, QTLMGRLEDGSPR, TLMGRLEDGSPRT,
GRLEDGSPRTGQA, QAFKQTYAKFDAN, QTYAKFDANSHND,
AKFDANSHNDDAL, DALLKNYGLLYCF, ALLKNYGLLYCFR,
KNYGLLYCFRKDM, YGLLYCFRKDMDK, GLLYCFRKDMDKV,
LLYCFRKDMDKVE, YCFRKDMDKVETF, KDMDKVETFLRIV,
DKVETFLRIVQCR, ETFLRIVQCRSVE, TFLRIVQCRSVEG,
LRIVQCRSVEGSC, RIVQCRSVEGSCG

Peptides are 13mers, amino acid are identified using single letter codes.

The results of a design and constructs according to step (c) and (d) of the above scheme and pertaining to the modified molecule of this invention is presented in Tables 2 and 3.

TABLE 2

Substitutions leading to the elimination of
T-cell epitopes of human hGH (WT = wild type
residue).

| | | |
|---|---|---|
| Residue | WT | |
| # | Residue | Substitution |

| | | | | | | | | | | | | | | |
|---|---|---|---|---|---|---|---|---|---|---|---|---|---|---|
| 4 | I | A | C | D | E | G | H | K | N | P | Q | R | S | T |
| 6 | L | A | C | D | E | G | H | K | N | P | Q | R | S | T |
| 9 | L | A | C | D | E | G | H | K | N | P | Q | R | S | T |
| 10 | F | A | C | D | E | G | H | K | N | P | Q | R | S | T |
| 14 | M | A | C | D | E | G | H | K | N | P | Q | R | S | T |
| 15 | L | A | C | D | E | G | H | K | N | P | Q | R | S | T |
| 20 | L | A | C | D | E | G | H | K | N | P | Q | R | S | T |
| 23 | L | A | C | D | E | G | H | K | N | P | Q | R | S | T |
| 25 | F | A | C | D | E | G | H | K | N | P | Q | R | S | T |
| 28 | Y | A | C | D | E | G | H | K | N | P | Q | R | S | T |
| 31 | F | A | C | D | E | G | H | K | N | P | Q | R | S | T |
| 35 | Y | A | C | D | E | G | H | K | N | P | Q | R | S | T |
| 36 | I | A | C | D | E | G | H | K | N | P | Q | R | S | T |
| 42 | Y | A | C | D | E | G | H | K | N | P | Q | R | S | T |
| 44 | F | A | C | D | E | G | H | K | N | P | Q | R | S | T |
| 45 | L | A | C | D | E | G | H | K | N | P | Q | R | S | T |
| 52 | L | A | C | D | E | G | H | K | N | P | Q | R | S | T |
| 54 | F | A | C | D | E | G | H | K | N | P | Q | R | S | T |
| 58 | I | A | C | D | E | G | H | K | N | P | Q | R | S | T |
| 73 | L | A | C | D | E | G | H | K | N | P | Q | R | S | T |
| 75 | L | A | C | D | E | G | H | K | N | P | Q | R | S | T |
| 76 | L | A | C | D | E | G | H | K | N | P | Q | R | S | T |
| 78 | I | A | C | D | E | G | H | K | N | P | Q | R | S | T |
| 80 | L | A | C | D | E | G | H | K | N | P | Q | R | S | T |
| 81 | L | A | C | D | E | G | H | K | N | P | Q | R | S | T |
| 82 | L | A | C | D | E | G | H | K | N | P | Q | R | S | T |
| 83 | I | A | C | D | E | G | H | K | N | P | Q | R | S | T |
| 86 | W | A | C | D | E | G | H | K | N | P | Q | R | S | T |
| 87 | L | A | C | D | E | G | H | K | N | P | Q | R | S | T |
| 90 | V | A | C | D | E | G | H | K | N | P | Q | R | S | T |
| 92 | F | A | C | D | E | G | H | K | N | P | Q | R | S | T |
| 93 | L | A | C | D | E | G | H | K | N | P | Q | R | S | T |
| 96 | V | A | C | D | E | G | H | K | N | P | Q | R | S | T |
| 97 | F | A | C | D | E | G | H | K | N | P | Q | R | S | T |
| 101 | L | A | C | D | E | G | H | K | N | P | Q | R | S | T |
| 102 | V | A | C | D | E | G | H | K | N | P | Q | R | S | T |
| 103 | Y | A | C | D | E | G | H | K | N | P | Q | R | S | T |
| 110 | V | A | C | D | E | G | H | K | N | P | Q | R | S | T |
| 111 | Y | A | C | D | E | G | H | K | N | P | Q | R | S | T |
| 113 | L | A | C | D | E | G | H | K | N | P | Q | R | S | T |
| 114 | L | A | C | D | E | G | H | K | N | P | Q | R | S | T |
| 117 | L | A | C | D | E | G | H | K | N | P | Q | R | S | T |
| 121 | I | A | C | D | E | G | H | K | N | P | Q | R | S | T |
| 124 | L | A | C | D | E | G | H | K | N | P | Q | R | S | T |
| 125 | M | A | C | D | E | G | H | K | N | P | Q | R | S | T |
| 128 | L | A | C | D | E | G | H | K | N | P | Q | R | S | T |
| 139 | F | A | C | D | E | G | H | K | N | P | Q | R | S | T |
| 143 | Y | A | C | D | E | G | H | K | N | P | Q | R | S | T |
| 146 | F | A | C | D | E | G | H | K | N | P | Q | R | S | T |
| 156 | L | A | C | D | E | G | H | K | N | P | Q | R | S | T |
| 157 | L | A | C | D | E | G | H | K | N | P | Q | R | S | T |
| 160 | Y | A | C | D | E | G | H | K | N | P | Q | R | S | T |
| 162 | L | A | C | D | E | G | H | K | N | P | Q | R | S | T |
| 163 | L | A | C | D | E | G | H | K | N | P | Q | R | S | T |
| 164 | Y | A | C | D | E | G | H | K | N | P | Q | R | S | T |
| 166 | F | A | C | D | E | G | H | K | N | P | Q | R | S | T |
| 170 | M | A | C | D | E | G | H | K | N | P | Q | R | S | T |
| 173 | V | A | C | D | E | G | H | K | N | P | Q | R | S | T |
| 176 | F | A | C | D | E | G | H | K | N | P | Q | R | S | T |
| 177 | L | A | C | D | E | G | H | K | N | P | Q | R | S | T |
| 179 | I | A | C | D | E | G | H | K | N | P | Q | R | S | T |
| 180 | V | A | C | D | E | G | H | K | N | P | Q | R | S | T |

TABLE 3

Additional substitutions leading to the removal
of a potential T-cell epitope for 1 or more
MHC allotypes.

| | | |
|---|---|---|
| | WT | |
| Residue | Residue | Substitution |

| | | | | | | | | | | | | | | | | | | | | |
|---|---|---|---|---|---|---|---|---|---|---|---|---|---|---|---|---|---|---|---|---|
| 6 | L | M | W | Y | | | | | | | | | | | | | | | | |
| 9 | L | I | M | V | W | Y | | | | | | | | | | | | | | |
| 11 | Q | H | | | | | | | | | | | | | | | | | | |
| 12 | N | A | C | G | P | T | | | | | | | | | | | | | | |
| 14 | M | F | I | V | W | Y | | | | | | | | | | | | | | |
| 15 | L | F | I | M | V | W | Y | | | | | | | | | | | | | |
| 16 | R | A | C | G | P | | | | | | | | | | | | | | | |
| 17 | A | D | E | H | K | N | P | Q | R | S | T | | | | | | | | | |
| 18 | H | P | | | | | | | | | | | | | | | | | | |
| 19 | R | A | C | G | P | | | | | | | | | | | | | | | |
| 20 | L | F | I | M | V | W | Y | | | | | | | | | | | | | |
| 21 | H | P | T | | | | | | | | | | | | | | | | | |
| 22 | Q | A | C | G | P | T | | | | | | | | | | | | | | |
| 23 | L | W | I | M | V | W | Y | | | | | | | | | | | | | |
| 25 | F | W | Y | | | | | | | | | | | | | | | | | |
| 26 | D | P | T | | | | | | | | | | | | | | | | | |
| 29 | E | T | | | | | | | | | | | | | | | | | | |
| 30 | E | H | | | | | | | | | | | | | | | | | | |
| 31 | F | I | V | W | | | | | | | | | | | | | | | | |
| 33 | E | H | T | | | | | | | | | | | | | | | | | |
| 34 | A | I | P | T | Y | | | | | | | | | | | | | | | |
| 36 | I | F | W | Y | | | | | | | | | | | | | | | | |
| 39 | E | T | | | | | | | | | | | | | | | | | | |
| 44 | F | W | Y | | | | | | | | | | | | | | | | | |
| 45 | L | F | I | M | W | Y | | | | | | | | | | | | | | |
| 47 | A | C | D | E | G | H | K | N | P | Q | R | S | T | | | | | | | |
| 49 | Q | D | H | | | | | | | | | | | | | | | | | |
| 50 | A | D | E | H | K | N | P | Q | R | S | T | | | | | | | | | |
| 51 | S | T | | | | | | | | | | | | | | | | | | |
| 52 | L | F | I | M | V | W | Y | | | | | | | | | | | | | |
| 53 | C | H | P | T | | | | | | | | | | | | | | | | |
| 73 | L | F | I | M | V | W | Y | | | | | | | | | | | | | |
| 74 | Q | A | C | G | P | | | | | | | | | | | | | | | |
| 75 | L | F | I | M | V | W | Y | | | | | | | | | | | | | |
| 76 | L | F | I | M | V | W | Y | | | | | | | | | | | | | |
| 77 | R | A | C | G | P | | | | | | | | | | | | | | | |
| 78 | I | W | Y | | | | | | | | | | | | | | | | | |
| 79 | S | A | C | G | P | T | | | | | | | | | | | | | | |
| 80 | L | F | I | M | V | W | Y | | | | | | | | | | | | | |
| 81 | L | F | I | M | V | W | Y | | | | | | | | | | | | | |
| 82 | L | F | I | M | V | W | Y | | | | | | | | | | | | | |
| 83 | I | F | W | Y | | | | | | | | | | | | | | | | |
| 84 | Q | A | C | G | P | T | | | | | | | | | | | | | | |
| 85 | S | A | C | G | P | | | | | | | | | | | | | | | |
| 86 | W | A | C | D | E | G | H | K | N | P | Q | R | S | T | | | | | | |
| 87 | L | A | C | D | E | G | H | K | M | N | P | Q | R | S | T | W | Y | | | |
| 88 | E | P | T | | | | | | | | | | | | | | | | | |
| 90 | V | M | W | Y | | | | | | | | | | | | | | | | |
| 93 | L | F | I | M | W | Y | | | | | | | | | | | | | | |
| 94 | R | A | C | G | P | | | | | | | | | | | | | | | |
| 95 | S | A | C | G | P | T | | | | | | | | | | | | | | |
| 96 | V | F | I | M | W | Y | | | | | | | | | | | | | | |
| 97 | F | M | W | Y | | | | | | | | | | | | | | | | |
| 98 | A | D | E | H | K | N | P | Q | R | S | T | | | | | | | | | |
| 99 | N | A | C | G | H | P | T | | | | | | | | | | | | | |
| 100 | S | A | C | G | P | T | | | | | | | | | | | | | | |
| 101 | L | F | I | M | V | W | Y | | | | | | | | | | | | | |
| 102 | V | F | I | M | W | Y | | | | | | | | | | | | | | |
| 104 | G | D | E | H | K | N | P | Q | R | S | T | | | | | | | | | |
| 105 | A | C | D | E | H | K | N | P | Q | R | S | T | | | | | | | | |
| 106 | S | A | C | D | G | H | P | | | | | | | | | | | | | |
| 107 | D | A | C | G | P | T | | | | | | | | | | | | | | |
| 108 | S | A | C | G | P | T | | | | | | | | | | | | | | |
| 109 | D | A | C | G | H | P | T | | | | | | | | | | | | | |
| 110 | V | M | W | Y | | | | | | | | | | | | | | | | |
| 112 | D | A | C | G | P | | | | | | | | | | | | | | | |
| 113 | L | F | I | M | V | W | Y | | | | | | | | | | | | | |
| 114 | L | F | I | M | V | W | Y | | | | | | | | | | | | | |
| 115 | K | A | C | G | H | P | | | | | | | | | | | | | | |
| 116 | D | A | C | G | H | P | T | | | | | | | | | | | | | |
| 117 | L | F | I | M | W | Y | | | | | | | | | | | | | | |
| 118 | E | A | C | G | H | P | T | | | | | | | | | | | | | |
| 119 | E | H | P | T | | | | | | | | | | | | | | | | |
| 120 | G | D | E | H | K | N | P | Q | R | S | T | | | | | | | | | |
| 121 | I | M | W | Y | | | | | | | | | | | | | | | | |
| 122 | Q | A | C | G | H | P | T | | | | | | | | | | | | | |
| 123 | T | A | C | G | P | | | | | | | | | | | | | | | |
| 124 | L | F | I | V | W | Y | | | | | | | | | | | | | | |
| 125 | M | F | I | V | W | Y | | | | | | | | | | | | | | |
| 126 | G | D | E | H | K | N | P | Q | S | | | | | | | | | | | |
| 127 | R | A | C | G | P | T | | | | | | | | | | | | | | |
| 128 | L | F | I | M | V | W | Y | | | | | | | | | | | | | |
| 129 | E | A | C | G | P | T | | | | | | | | | | | | | | |
| 130 | D | H | P | | | | | | | | | | | | | | | | | |
| 131 | G | C | D | E | H | K | N | P | Q | R | S | T | | | | | | | | |
| 132 | S | A | C | G | P | | | | | | | | | | | | | | | |
| 133 | P | T | | | | | | | | | | | | | | | | | | |
| 134 | R | P | T | | | | | | | | | | | | | | | | | |
| 135 | T | A | C | G | P | | | | | | | | | | | | | | | |
| 136 | G | H | P | T | | | | | | | | | | | | | | | | |
| 139 | F | M | W | Y | | | | | | | | | | | | | | | | |
| 140 | K | A | C | G | P | | | | | | | | | | | | | | | |
| 141 | Q | A | C | G | P | | | | | | | | | | | | | | | |
| 142 | T | P | | | | | | | | | | | | | | | | | | |
| 143 | Y | W | | | | | | | | | | | | | | | | | | |
| 144 | A | D | E | H | K | N | P | Q | R | S | T | | | | | | | | | |
| 145 | K | P | T | | | | | | | | | | | | | | | | | |
| 147 | D | P | T | | | | | | | | | | | | | | | | | |
| 148 | A | D | E | H | K | N | P | Q | R | S | T | | | | | | | | | |
| 149 | N | A | C | G | P | T | | | | | | | | | | | | | | |
| 151 | H | P | T | | | | | | | | | | | | | | | | | |
| 156 | L | F | I | M | V | W | Y | | | | | | | | | | | | | |
| 157 | L | F | I | M | W | Y | | | | | | | | | | | | | | |
| 158 | K | A | C | G | P | | | | | | | | | | | | | | | |
| 159 | N | A | C | G | H | P | T | | | | | | | | | | | | | |
| 161 | G | D | E | H | K | N | P | Q | S | | | | | | | | | | | |
| 162 | L | F | I | M | V | W | Y | | | | | | | | | | | | | |
| 163 | L | F | I | V | W | Y | | | | | | | | | | | | | | |
| 165 | C | D | E | H | K | N | P | Q | R | S | T | | | | | | | | | |
| 167 | R | A | C | G | H | P | | | | | | | | | | | | | | |
| 168 | K | H | P | S | T | | | | | | | | | | | | | | | |
| 169 | D | A | C | G | P | | | | | | | | | | | | | | | |
| 170 | M | I | V | W | Y | | | | | | | | | | | | | | | |
| 171 | D | A | C | G | P | | | | | | | | | | | | | | | |
| 173 | V | M | W | Y | | | | | | | | | | | | | | | | |
| 175 | T | H | | | | | | | | | | | | | | | | | | |
| 176 | F | W | Y | | | | | | | | | | | | | | | | | |
| 177 | L | I | M | W | Y | | | | | | | | | | | | | | | |
| 178 | R | H | P | T | | | | | | | | | | | | | | | | |
| 179 | I | W | Y | | | | | | | | | | | | | | | | | |
| 180 | V | F | I | M | W | Y | | | | | | | | | | | | | | |
| 181 | Q | A | C | G | P | | | | | | | | | | | | | | | |
| 182 | C | F | H | L | P | T | W | Y | | | | | | | | | | | | |
| 183 | R | I | P | T | V | Y | | | | | | | | | | | | | | |
| 184 | S | A | C | D | F | G | H | I | L | M | P | T | V | W | Y | | | | | |
| 185 | V | A | C | D | E | F | G | H | I | K | L | M | N | P | Q | R | S | T | W | Y |
| 186 | E | I | P | T | Y | | | | | | | | | | | | | | | |
| 187 | G | F | H | I | P | T | V | W | Y | | | | | | | | | | | |
| 188 | S | F | I | P | T | V | W | Y | | | | | | | | | | | | |

A further technical approach to the detection of T-cell epitopes is via biological T-cell assay. For the detection of T-cell epitopes within the hGH molecule a particularly effective method would be to test all or any of the peptide sequences of Table 1 for their ability to evoke an proliferative response in human T-cells cultured in vitro. The preferred method would be to exploit peripheral blood mononuclear cells (PBMC) from individuals where, in effect, the hGH protein antigen due to the nature of the genetic deficit in the individuals may constitute a foreign protein. In this sense, the protein is most likely to represent a potent antigen in vivo. This can be achieved using T cells subjected to several rounds of antigen (hGH) stimulation in vitro followed immediately by expansion in the presence of IL-2. For establishing polyclonal T cell lines 2-3 rounds of antigen stimulation are generally sufficient to generate a large number of antigen specific cells. These are used to screen large numbers of synthetic peptides (for example in the form of peptide pools), and they may be cryogenically stored to be used at a later date. After the initial round of antigen stimulation comprising co-incubation of the hGH antigen and PBMC for 7 days subsequent re-challenges with antigen are performed in the presence of most preferably autologous irradiated PBMC as antigen presenting cells. These rounds of antigen selection are performed for 3-4 days and are interspersed by expansion phases comprising stimulation with IL-2 which may be added every 3 days for a total period of around 9 days. The final re-challenge is performed using T-cells that have been “rested”, that is T cells which have not been IL-2 stimulated for around 4 days. These cells are stimulated with antigen (e.g. synthetic peptide or whole protein) using most preferably autologous antigen presenting cells as previously for around 4 days and the subsequent proliferative response (if any) is measured thereafter. The proliferative response can be measured by any convenient means and a widely known method for example would be to use an ³H-thymidine incorporation assay.

Accordingly the method embodied herein above comprises the production of T-cell lines or oligoclonal cultures derived from PBMC samples taken from individuals in whom previous therapeutic replacement therapy with hGH has been initiated to and in whom the replacement therapy has resulted in the induction of an immune response to the therapeutic protein. The lines or cultures from such individuals, are contacted with preparations of synthetic peptides or whole proteins and any in vitro the proliferative effects are measured. For any of the individual synthetic peptides or proteins, variants may be produced and re-tested for a continued ability to promote a significant proliferative response in the T-cell lines or cultures. Thus for example synthetic peptides containing any of the substitutions or combination of substitutions identified in Table 2 or Table 3 may be tested in such an assay.

Under this scheme it could be expected that the epitope map of the the hGH protein defined by the T-cell repertoire of a significant number of these individuals will be representative of the most prevalent peptide epitopes that are capable of presentation in the in vivo context. In this sense, PBMC from patients in whom there is a previously demonstrated immune response constitute the products of an in vivo priming step and given that the use of PBMC cell lines from such individuals is in principle an immunological in vitro recall assay, it further provides the practical benefit of there being the capacity for a much larger magnitude of proliferative response to any given stimulating peptide or protein. This reduces the technical challenge of conducting a proliferation measurement and in such a situation may give the opportunity for definition of a possible hierarchy of immunodominant epitopes as is the case for hGH which is demonstrated herein computationally to harbour multiple MHC class II peptide ligands and therefore multiple or complex (i.e. overlapping) T-cell epitopes.

Whilst it is particularly useful to establish T-cell lines of oligoclonal cultures from individuals in whom previous therapeutic hGH replacement therapy has resulted in the induction of an immune response to hGH, these are not the only source of cells which can be used to map the in vivo related immunogenic epitopes. Assay of naïve T-cells taken from healthy donors can equally be used, however in such an instance the magnitude of the stimulation index scored for any individual peptide is likely to be low requiring sensitive measurement to discern the peptide or protein induced stimulation from that of the background. The inventors have established in the operation of such an assay using well known techniques that a stimulation index equal to or greater than 2.0 is a useful measure of induced proliferation where the stimulation index is derived by division of the proliferation score measured (e.g. counts per minute if using ³H-thymidine incorporation) to the test (poly) peptide by the proliferation score measured in cells not contacted with a test (poly)peptide. A suitable method of this type is detailed in Example 2.

Where multiple potential epitopes are identified and in particular where a number of peptide sequences are found to be able to stimulate T-cells in a biological assay, cognisance may also be made of the structural features of the protein in relation to its propensity to evoke an immune response via the MHC class II presentation pathway. For example where the crystal structure of the protein of interest is known the crystallographic B-factor score may be analysed for evidence of structural disorder within the protein, a parameter suggested to correlate with the proximity to the biologically relevant immunodominant peptide epitopes [Dai G. et al (2001) J. Biological Chem. 276: 41913-41920]. Such an analysis when conducted on the hGH crystal structures [PDB ID:1 HGU Chantalat, L. et al (1995), Protein And Peptide Letters 2:333 & PDB ID 1A22 Clackson, T. et al (1998), J. Mol. Biol. 277: 1111] suggests a high likelihood for multiple immunodominant epitopes with at least 7 peaks of above mean B-factor scores within the non-receptor bound structure [PDB ID 1HGU]. This analysis indicates that the biologically relevant T-cell epitopes map to regions in the hGH sequence downstream from glutamine residue 41. Accordingly, under the scheme of the present; of the amino acid substitutions listed in Table 2 and Table 3, the most preferred substitutions comprise those directed to residues encompassed within residue numbers 42-180.

In practice a number of variant hGH proteins will be produced and tested for the desired immune and functional characteristic. Reference can be made to the mutations in the published literature known to result in alteration of the functional characteristics of the molecule [Lowman H. B. & Wells J. A. (1993) J. Mol. Biol. 243: 564-578; Wells J. A. et al (1993) Recent Prog. Horm. Res. 48: 253-275] and those substitutions listed in Table 2 and Table 3 which are also known to be deleterious to the protein function may be excluded for analysis or alternatively compensatory mutation may be conducted in order to restore functional activity of the protein. In all instances the variant proteins will most preferably be produced by the widely known methods of recombinant DNA technology although other procedures including chemical synthesis of hGH fragments may be contemplated. The invention relates to hGH analogues in which substitutions of at least one amino acid residue have been made at positions resulting in a substantial reduction in activity of or elimination of one or more potential T-cell epitopes from the protein. It is most preferred to provide hGH molecules in which amino acid modification (e.g. a substitution) is conducted within the most immunogenic regions of the parent molecule. The major preferred embodiments of the present invention comprise hGH molecules for which any of the MHC class II ligands are altered such as to eliminate binding or otherwise reduce the numbers of MHC allotypes to which the peptide can bind.

For the elimination of T-cell epitopes, amino acid substitutions are preferably made at appropriate points within the peptide sequence predicted to achieve substantial reduction or elimination of the activity of the T-cell epitope. In practice an appropriate point will preferably equate to an amino acid residue binding within one of the pockets provided within the MHC class II binding groove.

It is most preferred to alter binding within the first pocket of the cleft at the so-called P1 or P1 anchor position of the peptide. The quality of binding interaction between the P1 anchor residue of the peptide and the first pocket of the MHC class II binding groove is recognized as being a major determinant of overall binding affinity for the whole peptide. An appropriate substitution at this position of the peptide will be for a residue less readily accommodated within the pocket, for example, substitution to a more hydrophilic residue. Amino acid residues in the peptide at positions equating to binding within other pocket regions within the MHC binding cleft are also considered and fall under the scope of the present.

It is understood that single amino acid substitutions within a given potential T-cell epitope are the most preferred route by which the epitope may be eliminated. Combinations of substitution within a single epitope may be contemplated and for example can be particularly appropriate where individually defined epitopes are in overlap with each other. Moreover, amino acid substitutions either singly within a given epitope or in combination within a single epitope may be made at positions not equating to the “pocket residues” with respect to the MHC class II binding groove, but at any point within the peptide sequence. Substitutions may be made with reference to an homologues structure or structural method produced using in silico techniques known in the art and may be based on known structural features of the molecule according to this invention. All such substitutions fall within the scope of the present invention.

Amino acid substitutions other than within the peptides identified herein may be contemplated particularly when made in combination with substitution(s) made within a listed peptide. For example a change may be contemplated to restore structure or biological activity of the variant molecule. Such compensatory changes and changes to include deletion or addition of particular amino acid residues from the hGH polypeptide resulting in a variant with desired activity and in combination with changes in any of the disclosed peptides fall under the scope of the present.

In as far as this invention relates to modified hGH, compositions containing such modified hGH proteins or fragments of modified hGH proteins and related compositions should be considered within the scope of the invention. In another aspect, the present invention relates to nucleic acids encoding modified hGH entities. In a further aspect the present invention relates to methods for therapeutic treatment of humans using the modified hGH proteins.

In a further aspect still, the invention relates to methods for therapeutic treatment using pharmaceutical preparations comprising peptide or derivative molecules with sequence identity or part identity with the sequences herein disclosed.

### EXAMPLE 1

There are a number of factors that play important roles in determining the total structure of a protein or polypeptide. First, the peptide bond, i.e., that bond which joins the amino acids in the chain together, is a covalent bond. This bond is planar in structure, essentially a substituted amide. An “amide” is any of a group of organic compounds containing the grouping —CONH—.

The planar peptide bond linking Cα of adjacent amino acids may be represented as depicted below:

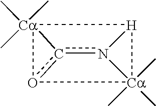

Because the O═C and the C—N atoms lie in a relatively rigid plane, free rotation does not occur about these axes. Hence, a plane schematically depicted by the interrupted line is sometimes referred to as an “amide” or “peptide plane” plane wherein lie the oxygen (O), carbon (C), nitrogen (N), and hydrogen (H) atoms of the peptide backbone. At opposite corners of this amide plane are located the Cα atoms. Since there is substantially no rotation about the O═C and C—N atoms in the peptide or amide plane, a polypeptide chain thus comprises a series of planar peptide linkages joining the Cα atoms. A second factor that plays an important role in defining the total structure or conformation of a polypeptide or protein is the angle of rotation of each amide plane about the common Cα linkage. The terms “angle of rotation” and “torsion angle” are hereinafter regarded as equivalent terms. Assuming that the O, C, N, and H atoms remain in the amide plane (which is usually a valid assumption, although there may be some slight deviations from planarity of these atoms for some conformations), these angles of rotation define the N and R polypeptide's backbone conformation, i.e., the structure as it exists between adjacent residues. These two angles are known as φ and ψ. A set of the angles φ₁, ψ₁, where the subscript i represents a particular residue of a polypeptide chain, thus effectively defines the polypeptide secondary structure. The conventions used in defining the φ, ψ angles, i.e., the reference points at which the amide planes form a zero degree angle, and the definition of which angle is φ, and which angle is ψ, for a given polypeptide, are defined in the literature. See, e.g,, Ramachandran et al. Adv. Prot. Chem. 23:283-437 (1968), at pages 285-94, which pages are incorporated herein by reference.

The present method can be applied to any protein, and is based in part upon the discovery that in humans the primary Pocket 1 anchor position of MHC Class II molecule binding grooves has a well designed specificity for particular amino acid side chains. The specificity of this pocket is determined by the identity of the amino acid at position 86 of the beta chain of the MHC Class II molecule. This site is located at the bottom of Pocket 1 and determines the size of the side chain that can be accommodated by this pocket.

Marshall, K. W., J. Immunol., 152:4946-4956 (1994). If this residue is a glycine, then all hydrophobic aliphatic and aromatic amino acids (hydrophobic aliphatics being: valine, leucine, isoleucine, methionine and aromatics being: phenylalanine, tyrosine and tryptophan) can be accommodated in the pocket, a preference being for the aromatic side chains. If this pocket residue is a valine, then the side chain of this amino acid protrudes into the pocket and restricts the size of peptide side chains that can be accommodated such that only hydrophobic aliphatic side chains can be accommodated. Therefore, in an amino acid residue sequence, wherever an amino acid with a hydrophobic aliphatic or aromatic side chain is found, there is the potential for a MHC Class II restricted T-cell epitope to be present. If the side-chain is hydrophobic aliphatic, however, it is approximately twice as likely to be associated with a T-cell epitope than an aromatic side chain (assuming an approximately even distribution of Pocket 1 types throughout the global population).

A computational method embodying the present invention profiles the likelihood of peptide regions to contain T-cell epitopes as follows:
(1) The primary sequence of a peptide segment of predetermined length is scanned, and all hydrophobic aliphatic and aromatic side chains present are identified. (2)The hydrophobic aliphatic side chains are assigned a value greater than that for the aromatic side chains; preferably about twice the value assigned to the aromatic side chains, e.g., a value of 2 for a hydrophobic aliphatic side chain and a value of 1 for an aromatic side chain. (3) The values determined to be present are summed for each overlapping amino acid residue segment (window) of predetermined uniform length within the peptide, and the total value for a particular segment (window) is assigned to a single amino acid residue at an intermediate position of the segment (window), preferably to a residue at about the midpoint of the sampled segment (window). This procedure is repeated for each sampled overlapping amino acid residue segment (window). Thus, each amino acid residue of the peptide is assigned a value that relates to the likelihood of a T-cell epitope being present in that particular segment (window). (4) The values calculated and assigned as described in Step 3, above, can be plotted against the amino acid coordinates of the entire amino acid residue sequence being assessed. (5) All portions of the sequence which have a score of a predetermined value, e.g., a value of 1, are deemed likely to contain a T-cell epitope and can be modified, if desired.

This particular aspect of the present invention provides a general method by which the regions of peptides likely to contain T-cell epitopes can be described. Modifications to the peptide in these regions have the potential to modify the MHC Class II binding characteristics.

According to another aspect of the present invention, T-cell epitopes can be predicted with greater accuracy by the use of a more sophisticated computational method which takes into account the interactions of peptides with models of MHC Class II alleles. The computational prediction of T-cell epitopes present within a peptide according to this particular aspect contemplates the construction of models of at least 42 MHC Class II alleles based upon the structures of all known MHC Class II molecules and a method for the use of these models in the computational identification of T-cell epitopes, the construction of libraries of peptide backbones for each model in order to allow for the known variability in relative peptide backbone alpha carbon (Cα) positions, the construction of libraries of amino-acid side chain conformations for each backbone dock with each model for each of the 20 amino-acid alternatives at positions critical for the interaction between peptide and MHC Class II molecule, and the use of these libraries of backbones and side-chain conformations in conjunction with a scoring function to select the optimum backbone and side-chain conformation for a particular peptide docked with a particular MHC Class II molecule and the derivation of a binding score from this interaction.

Models of MHC Class II molecules can be derived via homology modeling from a number of similar structures found in the Brookhaven Protein Data Bank (“PDB”). These may be made by the use of semi-automatic homology modeling software (Modeller, Sali A. & Blundell TL., 1993. J. Mol Biol 234:779-815) which incorporates a simulated annealing function, in conjunction with the CHARMm force-field for energy minimisation (available from Molecular Simulations Inc., San Diego, Calif.). Alternative modeling methods can be utilized as well.

The present method differs significantly from other computational methods which use libraries of experimentally derived binding data of each amino-acid alternative at each position in the binding groove for a small set of MHC Class II molecules (Marshall, K. W., et al., Biomed. Pept. Proteins Nucleic Acids, 1(3):157-162) (1995) or yet other computational methods which use similar experimental binding data in order to define the binding characteristics of particular types of binding pockets within the groove, again using a relatively small subset of MHC Class II molecules, and then ‘mixing and matching’ pocket types from this pocket library to artificially create further ‘virtual’ MHC Class II molecules (Sturniolo T., et al., Nat. Biotech, 17(6): 555-561 (1999). Both prior methods suffer the major disadvantage that, due to the complexity of the assays and the need to synthesize large numbers of peptide variants, only a small number of MHC Class II molecules can be experimentally scanned. Therefore the first prior method can only make predictions for a small number of MHC Class II molecules. The second prior method also makes the assumption that a pocket lined with similar amino-acids in one molecule will have the same binding characteristics when in the context of a different Class II allele and suffers further disadvantages in that only those MHC Class II molecules can be ‘virtually’ created which contain pockets contained within the pocket library. Using the modeling approach described herein, the structure of any number and type of MHC Class II molecules can be deduced, therefore alleles can be specifically selected to be representative of the global population. In addition, the number of MHC Class II molecules scanned can be increased by making further models further than having to generate additional data via complex experimentation. The use of a backbone library allows for variation in the positions of the Cα atoms of the various peptides being scanned when docked with particular MHC Class II molecules. This is again in contrast to the alternative prior computational methods described above which rely on the use of simplified peptide backbones for scanning amino-acid binding in particular pockets. These simplified backbones are not likely to be representative of backbone conformations found in ‘real’ peptides leading to inaccuracies in prediction of peptide binding. The present backbone library is created by superposing the backbones of all peptides bound to MHC Class II molecules found within the Protein Data Bank and noting the root mean square (RMS) deviation between the Cα atoms of each of the eleven amino-acids located within the binding groove. While this library can be derived from a small number of suitable available mouse and human structures (currently 13), in order to allow for the possibility of even greater variability, the RMS figure for each C″-α position is increased by 50%. The average Cα position of each amino-acid is then determined and a sphere drawn around this point whose radius equals the RMS deviation at that position plus 50%. This sphere represents all allowed Cα positions. Working from the Cα with the least RMS deviation (that of the amino-acid in Pocket 1 as mentioned above, equivalent to Position 2 of the 11 residues in the binding groove), the sphere is three-dimensionally gridded, and each vertex within the grid is then used as a possible location for a Cα of that amino-acid. The subsequent amide plane, corresponding to the peptide bond to the subsequent amino-acid is grafted onto each of these Cαs and the φ and ψ angles are rotated step-wise at set intervals in order to position the subsequent Cα. If the subsequent Cα falls within the ‘sphere of allowed positions’ for this Cα than the orientation of the dipeptide is accepted, whereas if it falls outside the sphere then the dipeptide is rejected.

This process is then repeated for each of the subsequent Cα positions, such that the peptide grows from the Pocket 1 Cα ‘seed’, until all nine subsequent Cαs have been positioned from all possible permutations of the preceding Cαs. The process is then repeated once more for the single Cα preceding pocket 1 to create a library of backbone Cα positions located within the binding groove.

The number of backbones generated is dependent upon several factors: The size of the ‘spheres of allowed positions’; the fineness of the gridding of the ‘primary sphere’ at the Pocket 1 position; the fineness of the step-wise rotation of the φ and ψ angles used to position subsequent Cαs. Using this process, a large library of backbones can be created. The larger the backbone library, the more likely it will be that the optimum fit will be found for a particular peptide within the binding groove of an MHC Class II molecule. Inasmuch as all backbones will not be suitable for docking with all the models of MHC Class II molecules due to clashes with amino-acids of the binding domains, for each allele a subset of the library is created comprising backbones which can be accommodated by that allele.

The use of the backbone library, in conjunction with the models of MHC Class II molecules creates an exhaustive database consisting of allowed side chain conformations for each amino-acid in each position of the binding groove for each MHC Class II molecule docked with each allowed backbone. This data set is generated using a simple steric overlap function where a MHC Class II molecule is docked with a backbone and an amino-acid side chain is grafted onto the backbone at the desired position. Each of the rotatable bonds of the side chain is rotated step-wise at set intervals and the resultant positions of the atoms dependent upon that bond noted. The interaction of the atom with atoms of side-chains of the binding groove is noted and positions are either accepted or rejected according to the following criteria: The sum total of the overlap of all atoms so far positioned must not exceed a pre-determined value. Thus the stringency of the conformational search is a function of the interval used in the step-wise rotation of the bond and the pre-determined limit for the total overlap. This latter value can be small if it is known that a particular pocket is rigid, however the stringency can be relaxed if the positions of pocket side-chains are known to be relatively flexible. Thus allowances can be made to imitate variations in flexibility within pockets of the binding groove. This conformational search is then repeated for every amino-acid at every position of each backbone when docked with each of the MHC Class II molecules to create the exhaustive database of side-chain conformations.

A suitable mathematical expression is used to estimate the energy of binding between models of MHC Class II molecules in conjunction with peptide ligand conformations which have to be empirically derived by scanning the large database of backbone/side-chain conformations described above. Thus a protein is scanned for potential T-cell epitopes by subjecting each possible peptide of length varying between 9 and 20 amino-acids (although the length is kept constant for each scan) to the following computations: An MHC Class II molecule is selected together with a peptide backbone allowed for that molecule and the side-chains corresponding to the desired peptide sequence are grafted on. Atom identity and interatomic distance data relating to a particular side-chain at a particular position on the backbone are collected for each allowed conformation of that amino-acid (obtained from the database described above). This is repeated for each side-chain along the backbone and peptide scores derived using a scoring function. The best score for that backbone is retained and the process repeated for each allowed backbone for the selected model. The scores from all allowed backbones are compared and the highest score is deemed to be the peptide score for the desired peptide in that MHC Class II model. This process is then repeated for each model with every possible peptide derived from the protein being scanned, and the scores for peptides versus models are displayed.

In the context of the present invention, each ligand presented for the binding affinity calculation is an amino-acid segment selected from a peptide or protein as discussed above. Thus, the ligand is a selected stretch of amino acids about 9 to 20 amino acids in length derived from a peptide, polypeptide or protein of known sequence. The terms “amino acids” and “residues” are hereinafter regarded as equivalent terms.

The ligand, in the form of the consecutive amino acids of the peptide to be examined grafted onto a backbone from the backbone library, is positioned in the binding cleft of an MHC Class II molecule from the MHC Class II molecule model library via the coordinates of the C″-α atoms of the peptide backbone and an allowed conformation for each side-chain is selected from the database of allowed conformations. The relevant atom identities and interatomic distances are also retrieved from this database and used to calculate the peptide binding score. Ligands with a high binding affinity for the MHC Class II binding pocket are flagged as candidates for site-directed mutagenesis. Amino-acid substitutions are made in the flagged ligand (and hence in the protein of interest) which is then retested using the scoring function in order to determine changes which reduce the binding affinity below a predetermined threshold value. These changes can then be incorporated into the protein of interest to remove T-cell epitopes.

Binding between the peptide ligand and the binding groove of MHC Class II molecules involves non-covalent interactions including, but not limited to: hydrogen bonds, electrostatic interactions, hydrophobic (lipophilic) interactions and Van der Walls interactions. These are included in the peptide scoring function as described in detail below.

It should be understood that a hydrogen bond is a non-covalent bond which can be formed between polar or charged groups and consists of a hydrogen atom shared by two other atoms. The hydrogen of the hydrogen donor has a positive charge where the hydrogen acceptor has a partial negative charge. For the purposes of peptide/protein interactions, hydrogen bond donors may be either nitrogens with hydrogen attached or hydrogens attached to oxygen or nitrogen. Hydrogen bond acceptor atoms may be oxygens not attached to hydrogen, nitrogens with no hydrogens attached and one or two connections, or sulphurs with only one connection. Certain atoms, such as oxygens attached to hydrogens or imine nitrogens (e.g. C═NH may be both hydrogen acceptors or donors. Hydrogen bond energies range from 3 to 7 Kcal/mol and are much stronger than Van der Waal's bonds, but weaker than covalent bonds. Hydrogen bonds are also highly directional and are at their strongest when the donor atom, hydrogen atom and acceptor atom are co-linear.

Electrostatic bonds are formed between oppositely charged ion pairs and the strength of the interaction is inversely proportional to the square of the distance between the atoms according to Coulomb's law. The optimal distance between ion pairs is about 2.8 Å. In protein/peptide interactions, electrostatic bonds may be formed between arginine, histidine or lysine and aspartate or glutamate. The strength of the bond will depend upon the pKa of the ionizing group and the dielectric constant of the medium although they are approximately similar in strength to hydrogen bonds.

Lipophilic interactions are favorable hydrophobic-hydrophobic contacts that occur between he protein and peptide ligand. Usually, these will occur between hydrophobic amino acid side chains of the peptide buried within the pockets of the binding groove such that they are not exposed to solvent. Exposure of the hydrophobic residues to solvent is highly unfavorable since the surrounding solvent molecules are forced to hydrogen bond with each other forming cage-like clathrate structures. The resultant decrease in entropy is highly unfavorable. Lipophilic atoms may be sulphurs which are neither polar nor hydrogen acceptors and carbon atoms which are not polar.

Van der Waal's bonds are non-specific forces found between atoms which are 3-4 Å apart. They are weaker and less specific than hydrogen and electrostatic bonds. The distribution of electronic charge around an atom changes with time and, at any instant, the charge distribution is not symmetric. This transient asymmetry in electronic charge induces a similar asymmetry in neighboring atoms. The resultant attractive forces between atoms reaches a maximum at the Van der Waal's contact distance but diminishes very rapidly at about 1 Å to about 2 Å. Conversely, as atoms become separated by less than the contact distance, increasingly strong repulsive forces become dominant as the outer electron clouds of the atoms overlap. Although the attractive forces are relatively weak compared to electrostatic and hydrogen bonds (about 0.6 Kcal/mol), the repulsive forces in particular may be very important in determining whether a peptide ligand may bind successfully to a protein.

In one embodiment, the Böhm scoring function (SCORE1 approach) is used to estimate the binding constant. (Böhm, H. J., J. Comput Aided Mol. Des., 8(3):243-256 (1994) which is hereby incorporated in its entirety). In another embodiment, the scoring function (SCORE2 approach) is used to estimate the binding affinities as an indicator of a ligand containing a T-cell epitope (Böhm, H. J., J. Comput Aided Mol. Des., 12(4):309-323 (1998) which is hereby incorporated in its entirety). However, the Böhm scoring functions as described in the above references are used to estimate the binding affinity of a ligand to a protein where it is already known that the ligand successfully binds to the protein and the protein/ligand complex has had its structure solved, the solved structure being present in the Protein Data Bank (“PDB”). Therefore, the scoring function has been developed with the benefit of known positive binding data. In order to allow for discrimination between positive and negative binders, a repulsion term must be added to the equation. In addition, a more satisfactory estimate of binding energy is achieved by computing the lipophilic interactions in a pairwise manner rather than using the area based energy term of the above Böhm functions.

Therefore, in a preferred embodiment, the binding energy is estimated using a modified Böhm scoring function. In the modified Böhm scoring function, the binding energy between protein and ligand (ΔG_{bind}) is estimated considering the following parameters: The reduction of binding energy due to the overall loss of translational and rotational entropy of the ligand (ΔG₀); contributions from ideal hydrogen bonds (ΔG_{hb}) where at least one partner is neutral; contributions from unperturbed ionic interactions (ΔG_{ionic}); lipophilic interactions between lipophilic ligand atoms and lipophilic acceptor atoms (ΔGₗᵢₚₒ); the loss of binding energy due to the freezing of internal degrees of freedom in the ligand, i.e., the freedom of rotation about each C—C bond is reduced (ΔGᵣₒₜ); the energy of the interaction between the protein and ligand (E_{VdW}). Consideration of these terms gives eguation 1:
(ΔG_{bind})=(ΔG₀)+(ΔG_{hb}×N_{hb})+(ΔG_{ionic}×N_{ionic})+(ΔGₗᵢₚₒ×Nₗᵢₚₒ)+(ΔGᵣₒₜ+Nᵣₒₜ)+E_{VdW}).
Where N is the number of qualifying interactions for a specific term and, in one embodiment, ΔG₀, ΔG_{hb}, ΔG_{ionic}, ΔGₗᵢₚₒ and ΔGᵣₒₜ are constants which are given the values: 5.4, −4.7, −4.7, −0.17, and 1.4, respectively.

The term N_{hb} is calculated according to equation 2:
N_{hb}=Σ_{h-bonds}f(ΔR, Δα)×f(N_{neighb})×f_{pcs}
f(ΔR, Δα) is a penalty function which accounts for large deviations of hydrogen bonds from ideality and is calculated according to equation 3:
f(ΔR, Δ−α)=f1(ΔR)×f2(Δα)
Where:
f1(ΔR)=1 if ΔR<=TOL
or =1−(ΔR−TOL)/0.4 if ΔR<=0.4+TOL
or =0 if ΔR>0.4+TOL
   And:
f2(Δα)=1 if Δα<30°
or =1−(Δα−30)/50 if Δα<=80°
or =0 if Δα>80°
TOL is the tolerated deviation in hydrogen bond length=0.25 Å
ΔR is the deviation of the H—O/N hydrogen bond length from the ideal value=1.9 Å
Δα is the deviation of the hydrogen bond angle ∠_{N/O—H.,O/N} from its idealized value of 180°

f(N_{neighb)} distinguishes between concave and convex parts of a protein surface and therefore assigns greater weight to polar interactions found in pockets rather than those found at the protein surface. This function is calculated according to equation 4 below:
f(N_{neighb)}=(N_{neighb}/N_{neighb,0})^{α where α=}0.5
N_{neighb} is the number of non-hydrogen protein atoms that are closer than 5 Å to any given protein atom.
N_{neighb,0} is a constant=25
f_{pcs} is a function which allows for the polar contact surface area per hydrogen bond and therefore distinguishes between strong and weak hydrogen bonds and its value is determined according to the following criteria:
   f_{pcs}=β when Aₚₒₗₐᵣ/N_{HB}<10 Å²
   or f_{pcs}=1 when Aₚₒₗₐᵣ/N_{HB}>10 Å²
Aₚₒₗₐᵣ is the size of the polar protein-ligand contact surface
N_{HB} is the number of hydrogen bonds
β is a constant whose value=1.2

For the implementation of the modified Böhm scoring function, the contributions from ionic interactions, ΔG_{ionic}, are computed in a similar fashion to those from hydrogen bonds described above since the same geometry dependency is assumed.

The term Nₗᵢₚₒ is calculated according to equation 5 below:
Nₗᵢₚₒ=Σ_{1L}f(r_{1L}[t1])
f(r_{1L}) is calculated for all lipophilic ligand atoms, 1, and all lipophilic protein atoms, L, according to the following criteria:
f(r_{1L})=1 when r_{1L}<=R1f(r_{1L})=(r_{1L}−R1)/(R2−R1) when R2<r_{1L}>R1
f(r_{1L})=0 when r_{1L}>=R2
Where: R1=r₁^{vdw}+r_{L}^{vdw}+0.5
and R2=R1+3.0
and r₁^{vdw} is the Van der Waal's radius of atom 1
and r_{L}^{vdw} is the Van der Waal's radius of atom L

The term Nᵣₒₜ is the number of rotable bonds of the amino acid side chain and is taken to be the number of acyclic sp³sp³ and sp³-sp² bonds. Rotations of terminal —CH₃ or —NH₃ are not taken into account.

The final term, E_{VdW}, is calculated according to equation 6 below:
E_{VdW}=ε₁ε₂((r₁^{vdw}+r₂^{vdw)}¹²/r¹²−(r₁^{vdw}+r₂^{vdw)}⁶/r⁶),
where:
ε₁ and ε₂ are constants dependant upon atom identity
r₁^{vdw}+r₂^{vdw} are the Van der Waal's atomic radii
r is the distance between a pair of atoms.

With regard to Equation 6, in one embodiment, the constants ε₁ and ε₂ are given the atom values: C: 0.245, N: 0.283, O: 0.316, S: 0.316, respectively (i.e. for atoms of Carbon, Nitrogen, Oxygen and Sulphur, respectively). With regards to equations 5 and 6, the Van der Waal's radii are given the atom values C: 1.85, N: 1.75, O: 1.60, S: 2.00 Å.

It should be understood that all predetermined values and constants given in the equations above are determined within the constraints of current understandings of protein ligand interactions with particular regard to the type of computation being undertaken herein. Therefore, it is possible that, as this scoring function is refined further, these values and constants may change hence any suitable numerical value which gives the desired results in terms of estimating the binding energy of a protein to a ligand may be used and hence fall within the scope of the present invention.

As described above, the scoring function is applied to data extracted from the database of side-chain conformations, atom identities, and interatomic distances. For the purposes of the present description, the number of MHC Class II molecules included in this database is 42 models plus four solved structures. It should be apparent from the above descriptions that the modular nature of the construction of the computational method of the present invention means that new models can simply be added and scanned with the peptide backbone library and side-chain conformational search function to create additional data sets which can be processed by the peptide scoring function as described above. This allows for the repertoire of scanned MHC Class II molecules to easily be increased, or structures and associated data to be replaced if data are available to create more accurate models of the existing alleles.

The present prediction method can be calibrated against a data set comprising a large number of peptides whose affinity for various MHC Class II molecules has previously been experimentally determined. By comparison of calculated versus experimental data, a cut of value can be determined above which it is known that all experimentally determined T-cell epitopes are correctly predicted.

It should be understood that, although the above scoring function is relatively simple compared to some sophisticated methodologies that are available, the calculations are performed extremely rapidly. It should also be understood that the objective is not to calculate the true binding energy per se for each peptide docked in the binding groove of a selected MHC Class II protein. The underlying objective is to obtain comparative binding energy data as an aid to predicting the location of T-cell epitopes based on the primary structure (i.e. amino acid sequence) of a selected protein. A relatively high binding energy or a binding energy above a selected threshold value would suggest the presence of a T-cell epitope in the ligand. The ligand may then be subjected to at least one round of amino-acid substitution and the binding energy recalculated. Due to the rapid nature of the calculations, these manipulations of the peptide sequence can be performed interactively within the program's user interface on cost-effectively available computer hardware. Major investment in computer hardware is thus not required. It would be apparent to one skilled in the art that other available software could be used for the same purposes. In particular, more sophisticated software which is capable of docking ligands into protein binding-sites may be used in conjunction with energy minimization. Examples of docking software are: DOCK (Kuntz et al., J. Mol. Biol., 161:269-288 (1982)), LUDI (Böhm, H. J., J. Comput Aided Mol. Des., 8:623-632 (1994)) and FLEXX (Rarey M., et al., ISMB, 3:300-308 (1995)). Examples of molecular modeling and manipulation software include: AMBER (Tripos) and CHARMm (Molecular Simulations Inc.). The use of these computational methods would severely limit the throughput of the method of this invention due to the lengths of processing time required to make the necessary calculations. However, it is feasible that such methods could be used as a ‘secondary screen’ to obtain more accurate calculations of binding energy for peptides which are found to be ‘positive binders’ via the method of the present invention.

The limitation of processing time for sophisticated molecular mechanic or molecular dynamic calculations is one which is defined both by the design of the software which makes these calculations and the current technology limitations of computer hardware. It may be anticipated that, in the future, with the writing of more efficient code and the continuing increases in speed of computer processors, it may become feasible to make such calculations within a more manageable time-frame.

Further information on energy functions applied to macromolecules and consideration of the various interactions that take place within a folded protein structure can be found in: Brooks, B. R., et al., J. Comput. Chem., 4:187-217 (1983) and further information concerning general protein-ligand interactions can be found in: Dauber-Osguthorpe et al., Proteins4(1):3 1-47(1988), which are incorporated herein by reference in their entirety. Useful background information can also be found, for example, in Fasman, G. D., ed., Prediction of Protein Structure and the Principles of Protein Conformation, Plenum Press, New York, ISBN: 0-306 4313-9.

### EXAMPLE 2

### Method for Naïve T-cell Assay Using Synthetic Peptides

The interaction between MHC, peptide and T-cell receptor (TCR) provides the structural basis for the antigen specificity of T-cell recognition. T-cell proliferation assays test the binding of peptides to MHC and the recognition of MHC/peptide complexes by the TCR. In vitro T-cell proliferation assays of the present example, involve the stimulation of peripheral blood mononuclear cells (PBMCs), containing antigen presenting cells (APCs) and T-cells. Stimulation is conducted in vitro using synthetic peptide antigens, and in some experiments whole protein antigen. Stimulated T-cell proliferation is measured using ³H-thymidine (³H-Thy) and the presence of incorporated ³H-Thy assessed using scintillation counting of washed fixed cells.

Buffy coats from human blood stored for less than 12 hours are obtained from the National Blood Service (Addenbrooks Hospital, Cambridge, UK). Ficoll-paque is obtained from Amersham Pharmacia Biotech (Amersham, UK). Serum free AIM V media for the culture of primary human lymphocytes and containing L-glutamine, 50 μg/ml streptomycin, 10 μg/ml gentomycin and 0.1% human serum albumin is from Gibco-BRL (Paisley, UK). Synthetic peptides are obtained from Pepscan (The Netherlands) and Babraham Technix (Cambridge, UK).

Erythrocytes and leukocytes are separated from plasma and platelets by gentle centrifugation of buffy coats. The top phase (containing plasma and platelets) are removed and discarded. Erythrocytes and leukocytes are diluted 1:1 in phosphate buffered saline (PBS) and layered onto 15 ml ficoll-paque (Amersham Pharmacia, Amersham UK). Centrifugation is done according to the manufacturers recommended conditions and PBMCs harvested from the serum+PBS/ficoll paque interface. PBMCs are mixed with PBS (1:1) and collected by centrifugation. The supernatant is removed and discarded and the PBMC pellet resuspended in 50 ml PBS. Cells are again pelleted by centrifugation and the PBS supernatant discarded. Cells are resuspended using 50 ml AIM V media and at this point counted and viability assessed using trypan blue dye exclusion. Cells are again collected by centrifugation and the supernatant discarded. Cells are resuspended for cryogenic storage at a density of 3×10⁷ per ml. The storage medium is 90% (v/v) heat inactivated AB human serum (Sigma, Poole, UK) and 10% (v/v) DMSO (Sigma, Poole, UK). Cells are transferred to a regulated freezing container (Sigma) and placed at −70° C. overnight before transferring to liquid N₂ for long term storage. When required for use, cells are thawed rapidly in a water bath at 37° C. before transferring to 10 ml pre-warmed AIM V medium.

PBMC are stimulated with protein and peptide antigens in a 96 well flat bottom plate at a density of 2×10⁵ PBMC per well. PBMC are incubated for 7 days at 37° C. before pulsing with ³H-Thy (Amersham-Phamacia, Amersham, UK). For the present study, synthetic peptides (15 mers) which advance by 3 amino acid increments are generated that span the entire sequence of hGH or all or any of peptides from Table 1 or peptides containing substitutions detailed in Table 2 or Table 3 can be generated and used.

Each peptide is screened individually in triplicate against PBMC's isolated from 20 naïve donors. Two control peptides that have previously been shown to be immunogenic and a potent non-recall antigen KLH are used in each donor assay. The control antigens are as below:

| | |
|---|---|
| Peptide | Sequence |

| | |
|---|---|
| C-32 | Biotin-PKYVKQNTLKLAT |
| | Flu haemagglutinin 307-319 |
| C-49 | KVVDQIKKISKPVQH |
| | Chlamydia HSP 60 peptide |
| KLH | Whole protein from Keyhole Limpet |
| | Hemocyanin. |

Peptides are dissolved in DMSO to a final concentration of 10 mM, these stock solutions were then diluted 1/500 in AIM V media (final concentration 20 μM). Peptides were added to a flat bottom 96 well plate to give a final concentration of 2 and 20 μM in a 100 μl. The viability of thawed PBMC's was assessed by trypan blue dye exclusion, cells were then resuspended at a density of 2×10⁶ cells/ml, and 100 μl (2×10⁵ PBMC/well) was transferred to each well containing peptides. Triplicate well cultures are assayed at each peptide concentration. Plates are incubated for 7 days in a humidified atmosphere of 5% CO₂ at 37° C. Cells are pulsed for 18-21 hours with 1 μCi ³H-Thy/well before harvesting onto filter mats. CPM values are determined using a Wallac microplate beta top plate counter (Perkin Elmer) or similar. Results are expressed as stimulation indices, determined using the following formula:
Proliferation to test peptide CPM
Proliferation in untreated wells CPM

For a naïve T-cell assay of this kind, a stimulation index of greater than 2.0 is taken as a positive score. Where the same test peptide achieves a stimulation index of greater than 2.0 in more than on donor sample this is taken as evidence of likely a immunodominant epitope.

1. A modified molecule having the biological activity of human growth hormone (hGH) and being substantially non-immunogenic or less immunogenic than any non-modified molecule having the same biological activity when used in vivo.

2. A molecule according to claim 1, wherein said loss of immunogenicity is achieved by removing one or more T-cell epitopes derived from the originally non-modified molecule.

3. A molecule according to claim 1, wherein said loss of immunogenicity is achieved by reduction in numbers of MHC allotypes able to bind peptides derived from said molecule.

4-30. (cancelled).

31. An isolated protein that is homologous to human growth hormone, the human growth hormone having an amino acid sequence (SEQ ID NO: 1) that includes at least one T-cell epitope;
the protein having substantially the same amino acid sequence as SEQ ID NO: 1, but including at least one less T-cell epitope;
wherein the protein has substantially the same biological activity as human growth hormone, but is less immunogenic than said human growth hormone when both are exposed to the immune system of the same species.

32. The protein of claim 31 wherein the amino acid sequence of the protein includes one less T-cell epitope.

33. The protein of claim 31 wherein the amino acid sequence of the protein differs from SEQ ID NO: 1 by one to nine amino acid residues.

34. The protein of claim 31 wherein the amino acid sequence of the protein has at least one less amino acid residue than SEQ ID NO: 1.

35. The protein of claim 31 wherein the amino acid sequence of the protein has at least one more amino acid residue than SEQ ID NO: 1.

36. The protein of claim 31 wherein the amino acid sequence of the protein has the same number of amino acid residues as SEQ ID NO: 1.

37. The protein of claim 36 wherein the amino acid sequence of the protein differs from SEQ ID NO: 1 by one to nine amino acid residues.

38. The protein of claim 31 wherein the amino acid sequence of the protein contains at least one amino acid substitution in SEQ ID NO: 1 selected from the group of amino acid substitutions set forth in Table 2.

39. The protein of claim 38 wherein the amino acid sequence of the protein further contains at least one amino acid substitution in SEQ ID NO: 1 selected from the group of amino acid substitutions set forth in Table 3.

40. An isolated polypeptide having an amino acid sequence consisting of at least nine consecutive amino acid residues of a sequence selected from the group of sequences set forth in Table 1.

41. An isolated polypeptide having an amino acid sequence selected from the group of sequences set forth in Table 1.

42. An isolated polynucleotide encoding a protein of claim 31.

43. An isolated polynucleotide encoding a protein of claim 38.

44. An isolated polynucleotide encoding a protein of claim 39.

45. An isolated polynucleotide encoding a polypeptide of claim 41.

46. A method of preparing a protein of claim 31, the method comprising the steps of:
(i) identifying one or more potential T-cell epitopes within the amino acid sequence of human growth hormone (SEQ ID NO: 1);
(ii) selecting at least one sequence variant of at least one potential T-cell epitope identified in step (i) that eliminates or substantially reduces the MHC class II binding activity of the potential T-cell epitope; wherein the amino acid sequence of the selected variant differs from the amino acid sequence of the T-cell epitope identified in step (i) by at least one amino acid residue;
(iii) preparing, by recombinant DNA techniques, at least one protein that includes at least one variant selected in step (ii);
(iv) evaluating the biological activity and immunogenicity of at least one protein prepared in step (iii); and
(v) selecting a protein evaluated in step (iv) that has substantially the same biological activity as, but substantially less immunogenicity than human hormone.

47. The method of claim 46 wherein step (i) is carried out by determining the MHC class II binding affinity of potential T-cell epitope segments of human growth hormone using an in vitro assay, an in silico technique, or a biological assay.

48. The method of claim 46 wherein step (i) is carried out by:
(a) selecting a region of the amino acid sequence of human growth hormone (SEQ ID NO: 1);
(b) sequentially sampling overlapping amino acid residue segments of predetermined uniform size and including at least three amino acid residues from the selected region;
(c) calculating the MHC class II molecule binding score for each of the sampled segments by summing assigned values for each hydrophobic amino acid residue side chain present in the sampled amino acid residue segment; and
(d) identifying at least one segment that is suitable for modification based on the calculated MHC class II binding score for that segment to reduce the overall MHC class II binding score for the protein relative to the binding score for human growth hormone.

49. The method of claim 48 wherein step (c) is carried out by using a Böhm scoring function modified to include a van der Waal's ligand-protein energy repulsive term and a ligand conformational energy term by:
(1) selecting a model from a first database consisting of MHC class II molecule models;
(2) selecting an allowed peptide backbone from a second database consisting of allowed peptide backbones for the MHC class II molecule models in step (1);
(3) identifying amino acid residue side chains present in each sampled segment;
(4) determining the binding affinity value for all side chains present in each sampled segment; and
(5) repeating each of steps (1) through (4) for each model in the first database and for each backbone in the second database.

50. The method of claim 46 wherein step (ii) is carried out by substitution, addition, or deletion of one to nine amino acid residues from a potential T-cell epitope identified in step (i).

51. The protein of claim 31 having an amino acid sequence that is free from T-cell epitopes.

52. A protein prepared by the method of claim 46.

53. A pharmaceutical composition comprising a protein of claim 31 and a pharmaceutically acceptable carrier therefor.

54. A pharmaceutical composition comprising a protein of claim 38 and a pharmaceutically acceptable carrier therefor.

55. A pharmaceutical composition comprising a protein of claim 39 and a pharmaceutically acceptable carrier therefor.

56. A pharmaceutical composition comprising a protein of claim 51 and a pharmaceutically acceptable carrier therefor.

57. A pharmaceutical composition comprising a protein of claim 52 and a pharmaceutically acceptable carrier therefor.


SEQUENCE LISTING (65 sequences)

SEQ ID NO: 1 - protein, 191 aa, Homo sapiens
FPTIPLSRLFQNAMLRAHRLHQLAFDTYEEFEEAYIPKEQKYSFLQAPQASLCFSESIPTPSNREQAQQKSNLQLLRISLLLIQSWLEPVGFLRSVFANSLVYGASDSDVYDLLKDLEEGIQTLMGRLEDGSPRTGQAFKQTYAKFDANSHNDDALLKNYGLLYCFRKDMDKVETFLRIVQCRSVEGSCGF

SEQ ID NO: 2 - protein, 13 aa, Homo sapiens
PTIPLSRLFQNAM

SEQ ID NO: 3 - protein, 13 aa, Homo sapiens
IPLSRLFQNAMLR

SEQ ID NO: 4 - protein, 13 aa, Homo sapiens
SRLFQNAMLRAHR

SEQ ID NO: 5 - protein, 13 aa, Homo sapiens
RLFQNAMLRAHRL

SEQ ID NO: 6 - protein, 13 aa, Homo sapiens
NAMLRAHRLHQLA

SEQ ID NO: 7 - protein, 13 aa, Homo sapiens
AMLRAHRLHQLAF

SEQ ID NO: 8 - protein, 13 aa, Homo sapiens
HRLHQLAFDTYEE

SEQ ID NO: 9 - protein, 13 aa, Homo sapiens
HQLAFDTYEEFEE

SEQ ID NO: 10 - protein, 13 aa, Homo sapiens
LAFDTYEEFEEAY

SEQ ID NO: 11 - protein, 13 aa, Homo sapiens
DTYEEFEEAYIPK

SEQ ID NO: 12 - protein, 13 aa, Homo sapiens
EEFEEAYIPKEQK

SEQ ID NO: 13 - protein, 13 aa, Homo sapiens
EAYIPKEQKYSFL

SEQ ID NO: 14 - protein, 13 aa, Homo sapiens
AYIPKEQKYSFLQ

SEQ ID NO: 15 - protein, 13 aa, Homo sapiens
QKYSFLQAPQASL

SEQ ID NO: 16 - protein, 13 aa, Homo sapiens
YSFLQAPQASLCF

SEQ ID NO: 17 - protein, 13 aa, Homo sapiens
SFLQAPQASLCFS

SEQ ID NO: 18 - protein, 13 aa, Homo sapiens
ASLCFSESIPTPS

SEQ ID NO: 19 - protein, 13 aa, Homo sapiens
LCFSESIPTPSNR

SEQ ID NO: 20 - protein, 13 aa, Homo sapiens
ESIPTPSNREQAQ

SEQ ID NO: 21 - protein, 13 aa, Homo sapiens
SNLQLLRISLLLI

SEQ ID NO: 22 - protein, 13 aa, Homo sapiens
LQLLRISLLLIQS

SEQ ID NO: 23 - protein, 13 aa, Homo sapiens
QLLRISLLLIQSW

SEQ ID NO: 24 - protein, 13 aa, Homo sapiens
LRISLLLIQSWLE

SEQ ID NO: 25 - protein, 13 aa, Homo sapiens
ISLLLIQSWLEPV

SEQ ID NO: 26 - protein, 13 aa, Homo sapiens
SLLLIQSWLEPVG

SEQ ID NO: 27 - protein, 13 aa, Homo sapiens
LLLIQSWLEPVGF

SEQ ID NO: 28 - protein, 13 aa, Homo sapiens
LLIQSWLEPVGFL

SEQ ID NO: 29 - protein, 13 aa, Homo sapiens
QSWLEPVGFLRSV

SEQ ID NO: 30 - protein, 13 aa, Homo sapiens
SWLEPVGFLRSVF

SEQ ID NO: 31 - protein, 13 aa, Homo sapiens
EPVGFLRSVFANS

SEQ ID NO: 32 - protein, 13 aa, Homo sapiens
VGFLRSVFANSLV

SEQ ID NO: 33 - protein, 13 aa, Homo sapiens
GFLRSVFANSLVY

SEQ ID NO: 34 - protein, 13 aa, Homo sapiens
RSVFANSLVYGAS

SEQ ID NO: 35 - protein, 13 aa, Homo sapiens
SVFANSLVYGASD

SEQ ID NO: 36 - protein, 13 aa, Homo sapiens
NSLVYGASDSDVY

SEQ ID NO: 37 - protein, 13 aa, Homo sapiens
SLVYGASDSDVYD

SEQ ID NO: 38 - protein, 13 aa, Homo sapiens
LVYGASDSDVYDL

SEQ ID NO: 39 - protein, 13 aa, Homo sapiens
SDVYDLLKDLEEG

SEQ ID NO: 40 - protein, 13 aa, Homo sapiens
DVYDLLKDLEEGI

SEQ ID NO: 41 - protein, 13 aa, Homo sapiens
YDLLKDLEEGIQT

SEQ ID NO: 42 - protein, 13 aa, Homo sapiens
DLLKDLEEGIQTL

SEQ ID NO: 43 - protein, 13 aa, Homo sapiens
KDLEEGIQTLMGR

SEQ ID NO: 44 - protein, 13 aa, Homo sapiens
EGIQTLMGRLEDG

SEQ ID NO: 45 - protein, 13 aa, Homo sapiens
QTLMGRLEDGSPR

SEQ ID NO: 46 - protein, 13 aa, Homo sapiens
TLMGRLEDGSPRT

SEQ ID NO: 47 - protein, 13 aa, Homo sapiens
GRLEDGSPRTGQA

SEQ ID NO: 48 - protein, 13 aa, Homo sapiens
QAFKQTYAKFDAN

SEQ ID NO: 49 - protein, 13 aa, Homo sapiens
QTYAKFDANSHND

SEQ ID NO: 50 - protein, 13 aa, Homo sapiens
AKFDANSHNDDAL

SEQ ID NO: 51 - protein, 13 aa, Homo sapiens
DALLKNYGLLYCF

SEQ ID NO: 52 - protein, 13 aa, Homo sapiens
ALLKNYGLLYCFR

SEQ ID NO: 53 - protein, 13 aa, Homo sapiens
KNYGLLYCFRKDM

SEQ ID NO: 54 - protein, 13 aa, Homo sapiens
YGLLYCFRKDMDK

SEQ ID NO: 55 - protein, 13 aa, Homo sapiens
GLLYCFRKDMDKV

SEQ ID NO: 56 - protein, 13 aa, Homo sapiens
LLYCFRKDMDKVE

SEQ ID NO: 57 - protein, 13 aa, Homo sapiens
YCFRKDMDKVETF

SEQ ID NO: 58 - protein, 13 aa, Homo sapiens
KDMDKVETFLRIV

SEQ ID NO: 59 - protein, 13 aa, Homo sapiens
DKVETFLRIVQCR

SEQ ID NO: 60 - protein, 13 aa, Homo sapiens
ETFLRIVQCRSVE

SEQ ID NO: 61 - protein, 13 aa, Homo sapiens
TFLRIVQCRSVEG

SEQ ID NO: 62 - protein, 13 aa, Homo sapiens
LRIVQCRSVEGSC

SEQ ID NO: 63 - protein, 13 aa, Homo sapiens
RIVQCRSVEGSCG

SEQ ID NO: 64 - protein, 13 aa, Artificial Sequence
  Flu haemagglutinin fragment
PKYVKQNTLKLAT

SEQ ID NO: 65 - protein, 15 aa, Artificial Sequence
  Chlamydia fragment
KVVDQIKKISKPVQH